(19) United States
(12) Patent Application Publication
(10) Pub. No.: US 2002/0192182 A1
(43) Pub. Date: Dec. 19, 2002

(54) Polysaccharide-based polymerizable hydrogels

(76) Inventors: Stephen Massia, Mesa, AZ (US); Julie Trudel, Sunnyvale, CA (US); Julie-Anne M. Burdick, Gilbert, AZ (US)

Correspondence Address: DORITY & MANNING, P.A., POST OFFICE BOX 1449, GREENVILLE, SC, 29602-1449, US

(21) Appl. No.: 10/095,722

(22) Filed: Mar. 12, 2002

Related U.S. Application Data
(60) Provisional application No. 60/275,546, filed on Mar. 12, 2001.

(51) Int. Cl. A61K 31/765; A61K 31/785

(52) U.S. Cl. 424/78.32; 514/54

(57) ABSTRACT

A hydrogel is provided in which varying ratios of acrylyol-modified dextran molecules and acrylyol-modified hyaluronan products are cross-linked to form a hydrogel conjugate. The incorporation of the derivatized hyaluronan molecules allows a hydrogel to be constructed which may be degraded by hydrolysis and enzymatic pathways. This mechanism offers novel hydrogel which may be useful in medical applications including the prevention of surgical adhesions, controlled drug delivery, tissue coatings, tissue adherence, and tissue supporting structures, and the coating of medical devices and related articles prior to placement within a patient.

### RELATED APPLICATIONS

This application claims the benefit of US provisional application having serial No. 60/275,546 filed on Mar. 12, 2001, and which is incorporated herein by reference.

### FIELD OF THE INVENTION

This invention is directed towards an improved hydrogel which is useful as a biological carrier for pharmacological agents.

### BACKGROUND OF THE INVENTION

It is known in the art to provide biocompatible, biodegradable hydrogels which are prepared from derivatized backbone molecules bonded together using one or more cross-linking agents. The backbone molecules may include proteins, such as albumin, and polysaccharides, such as polymannuronic acid or polygalacturonic acid. Conventional derivatizing agents may include polyvalent derivatives of polyethylene or polyalkylene glycol.

One such hydrogel is taught in U.S. Pat. No. 5,514,379 assigned to General Hospital Corporation, Boston, Mass., and which is incorporated herein by reference. The hydrogel composition may be used as a carrier for diagnostic labels, therapeutic drugs such as antibiotics, or provide a localized environment for living cells that may produce therapeutic agents.

While a variety of hydrogels are known and used within the art, there remains room for improvement and variation within the art.

### SUMMARY OF THE INVENTION

The present invention is directed to a biodegradable and biocompatible hydrogel in which two different polysaccharides, such as dextran and hyaluronan, are used to form a single hydrogel.

It is an aspect of one of the present inventions to provide a novel hydrogel in which the rate of hydrogel degradation may be regulated by the relative proportions of the polysaccharide backbone component of the hydrogel.

It is yet another aspect of one of the present inventions to provide a hydrogel in which the rate of release of an associated pharmacological agent may be regulated by the relative proportions of a polysaccharide backbone component of the hydrogel.

It is yet a further aspect of one of the present inventions to provide a hydrogel having an increased number of available binding sites to which a pharmacological agent or other useful molecule may be bound. Such hydrogel may in accordance with one aspect of one of the inventions be supplied to a patient and upon the controlled hydrolosis of the hydrogel, the covalently bound material may be released and made available to the patient.

One or more of the aspects of one of the present inventions may be provided by a method for medical treatment comprising applying to tissue, cells, or medical devices an aqueous solution comprising a covalently polymerizable biodegradable polymer mixture, the mixture comprising a first derivatized polysaccharide of dextran and a second derivatized polysaccharide of hyaluronan; and, polymerizing the derivatized polymers onto a tissue, cell, or medical device wherein the derivatized dextran portion of the resulting gel comprises a water-soluble region and the derivatized hyaluronan providing a portion that can be degraded by enzymes.

Other aspects of one or more of the present inventions may be found in reference to a hydrogel having a matrix comprising a plurality of derivatized dextran molecules cross-linked with a plurality of derivatized hyaluronan molecules, wherein a weight ratio of the derivatized dextran molecules to the derivatized hyaluronan molecules ranges from about 20:80 to about 80:20.

These and other features, aspects, and advantages of the present invention will become better understood with reference to the following description and appended claims.

### BRIEF DESCRIPTION OF THE DRAWINGS

A full and enabling disclosure of the present invention, including the best mode thereof, to one of ordinary skill in the art, is set forth more particularly in the remainder of the specification, including reference to the accompanying drawings.

FIGS. 1A and 1B are schematic diagrams of a derivatized hydrogel backbone linked together by use of a cross-linking agent and at least one biological molecule entrapped (FIG. 1A) or covalently bound (FIG. 1B) to the hydrogel.

### DESCRIPTION OF THE PREFERRED EMBODIMENT

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers are used throughout to describe the same material, apparatus or process pathway. To avoid redundancy, detailed descriptions of much of the apparatus once described in relation to a figure is not repeated in the descriptions of subsequent figures, although such apparatus or process is labeled with the same reference numbers.

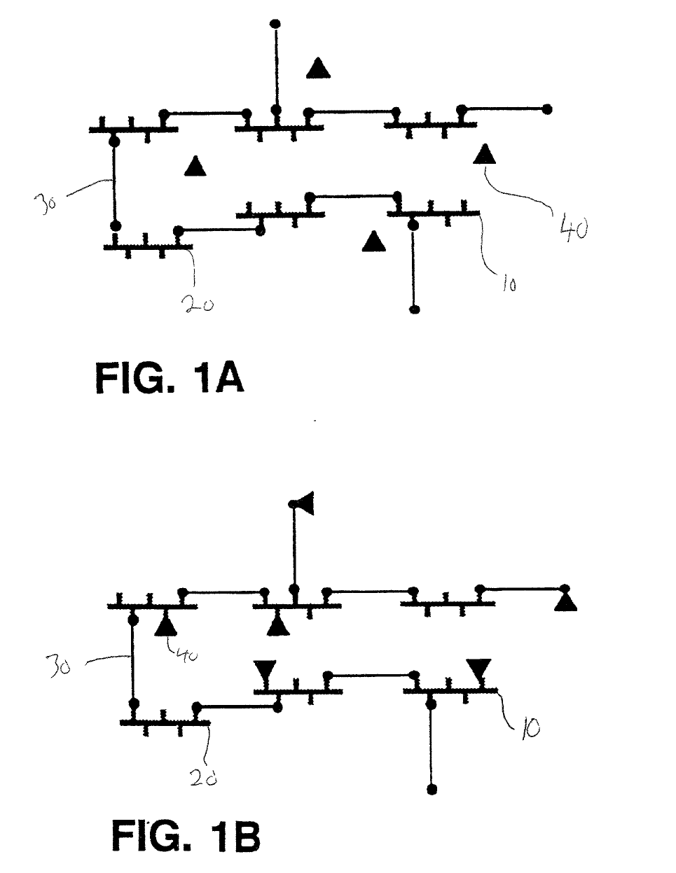

As seen in reference to FIGS. 1A & 1B, a hydrogel composition may be provided having backbone polysaccharides and which may include a mixture of a derivatized dextran backbone molecule 10 with a derivatized hyaluronan backbone 20 portion. The backbones are joined together by the action of the cross-linking agent 30 on the derivatized portions of the polysaccharide molecules. Carried within the hydrogel is at least one target molecule 40 which may be a pharmacologically active material such as an antibiotic or other drug.

In accordance with certain features of one or more of the inventions, it has been found that a useful hydrogel can be provided by controlling the relative proportions of the polysaccharide constituents of the hydrogel backbone. In so doing, the delivery rate or release of a drug/compound contained within the hydrogel may be varied. In general, a greater proportion of dextran within the hydrogel brings about a slower degradation of the resulting hydrogel. However, delivery rate is affected by a variety of factors including: 1) the ratio of derivatized dextran to derivatized hyaluronan; 2) the degree of substitution for each class molecules; 3) the amount of cross-linking as may be determined by time and the amount of cross-linking agents (initiators); and, 4) the molecular weight of the backbone polysaccharides.

### Hydrogel Synthesis

One methodology for the synthesis of derivatized polysaccharide-based materials, may be found in reference to van Dijk-Wolthuis, W. N. E. et al, A Synthesis, Characterization, and Polymerization of Glycidyl Methacrylate Derivatized Dextran, Macromolecules 28 (18):6317-6322 (1995) and which is incorporated herein by reference.

Dextran molecules may be modified with acryloyl functional groups according to the general reaction detailed in the van Dijk-Wolthuis reference cited above. Dextran, along with a catalyst, dimethylamino-pyridinine (DMAP) (Sigma Chemical Company), may be dissolved in DMSO (Mallinckrodt & Baker, Inc.) under nitrogen atmosphere and vigorous stirring at room temperature. Glycidylmethacrylete (GMA) (Sigma Chemical Company) is then added to the mixture to yield a derivatized dextran (acryloyl-dex). The degree of substitution (DS) may be controlled by the amount of GMA added to the mixture. In the example set forth below, a DS value of 15 was used for the derivatized dextran using the formula.

Dextran DS 15

25 g Dextran
225 ml DMSO
5 g DMAP
3.3 ml GMA

Following a 48 hour stirring interval, the catalyst is neutralized by adding an equi-molar amount of hydrochloric acid to the mixture.

The DMSO is subsequently removed from the acryloyl-dex mixture using dialysis tubing having a molecular weight cut off of 12,000 in combination with centrifugal filters (Centricon Plus—20, Millipore Corporation) using a swing-bucket centrifuge set at 4000 rpm. The resulting filtrate of acryloyl-dex is then collected and dissolved in de-ionized water. The solutions are then frozen and lyophilized to form a resulting powdered product. The lyophilized powder is stored in its powdered form.

The powdered acryloyl-dex may be used to form a hydrogel by dissolving the acryloyl-dex powder in a 25% solution of Dulbecco's Modified Eagle Medium (DMEM) (Life Technologies, Inc.). A free-radical polymerization reaction is initiated by adding photoinitiators to the solution followed by exposure to long wave ultra violet radiation ranging from 315 to 400 nm, with a peak at 365 nm. The photoinitiators used included a 30% solution of solid 2,2-dimethoxy-2-phenylacetophenone (Aldrich Chemical) mixed into 1-vinyl-2-pyrrolidinone (Aldrich Chemical). 3 microliters of photoinitiators were added for each milliliter of macromer solution. Exposure to the cross-linking ultra violet light lasted up to 5 minutes until hydrogels were formed.

The hydrogel cross-linking protocols used herein are similar to that developed with other diacrylate macromers as set forth in the publication of Sawhney et al, A Bioerodible Hydrogels Based on Photo Polymerized Poly (ethylene glycol)-Co-poly (α-hydroxy acid) Diacrylate Macromers. Macromolecules 1993 26(4): p. 581-587 which is incorporated herein by reference.

Hyaluronan hydrogels were also formed using the same free-radical photopolymerization reaction described above. Acryoyl functional groups were chemically added to the hyaluronan. Given the poor solubility of hyaluronan in DMSO, the reaction was conducted in an aqueous environment. The amount of GMA added to the solution was adjusted for a theoretical DS of 60. The high theoretical DS value was chosen since it is known that GMA is hydrophobic and the resulting reaction yield would be significantly lower than predicted based upon a molar calculation. The formula below was used:

Hyaluronic Acid, DS 60

200 ml dH₂O
0.5 g HyA
0.04 g DMAP
0.12 ml GMA

The acryloyl derivatization reaction was allowed to proceed for 48 hours and then stopped using an equi-molar addition of hydrochloric acid. The resulting mixture was dialyzed, frozen, and lyophilized as described above. The resulting acrylyol-hyaluronan (Acrylyol-Hya) powder was stored until used.

Hyaluronan hydrogels were formed using the same free-radial photopolymerization reaction as set forth above for dextran hydrogels.

### Extran/Hyaluronic Acid Conjugate Gel

The acrylyol-dex and the acrylyol-HyA powder products prepared above were also used to make a gel using the combined modified dextran and modified hyaluronan molecules. The acrylyol-dex and acrylyol-HyA powders were mixed at 20:80, 50:50, and 80:20 weight ratios by dissolving the powders in DMEM. The photoinitiators as set forth above were added to the mixture, and UV radiation as previously described was used to cross-link the molecules to form a hydrogel conjugate.

The hydrogel was evaluated as a delivery agent for other molecules of interest by incorporating into the hydrogel, prior to polymerization, RGD peptides (arginine, glycine, aspartic acid) which became physically entrapped within the hydrogel. As such, the hydrogel has the ability to contain within its matrix various added molecules. Accordingly, the hydrogel provides an effective delivery mechanism based upon the varying ratios of the backbone dextran and hyaluronan molecules, the degrees of substitution within each class of backbone molecules, the amount of cross-linking of the hydrogel, the degree of substitution of the hydrogel, along with the molecular weight of the polysaccharide backbone molecules.

The present hydrogel, having varying proportions of derivatized dextran and hyaluronan may be degraded by hydrolysis. This ability allows for a non-enzymatic release mechanism in addition to conventional enzymatic release and breakdown of the hyaluronan backbone polymer. The resulting hydrogel is derived from natural products, is biocompatible, and has been demonstrated as useful for the physical entrapment of bioactive peptide molecules. It is envisioned that a wide range of biomolecules may be incorporated into the hydrogel matrix. Further, the hydrogel is believed to have improved drug delivery capability in that dextran and hyaluronan provide multiple hydroxyl groups that are covalent binding sites for drugs and other biological molecules.

As set forth in the examples, one aspect of one of the inventions provides for an improved hydrogel having backbone polysaccharides of a dextran molecule and a hyaluronan molecule. The resulting hydrogel is believed useful for tissue engagement. For instance, the resulting hydrogel is useful in forming a biodegradable coating for reducing formation of surgical adhesions following a surgical procedure. The tissue surface may also be contacted with the hydrogel components which are then polymerized in situ forming a tissue junction. The ability to control the relative amount of hyaluronan (enzymatic degradation rate) in the hydrogel affords one the ability to determine how long an adhesive interval should occur for the hydrogel.

The hydrogel can also be used to form ultra thin, biodegradable tissue coatings such as along the lumen of a blood vessel. Further, the hydrogel can be used to provide a coating on a medical device or implement. One such example would be coating surfaces of a stent or catheter to allow for a longer useful life of the device. Further, the hydrogel can be used to create a tissue support by forming a shaped article within the body to serve a mechanical function. Such supports may include a sealant for a bleeding organ, a bone defect, or as a filler for a vascular aneurism. Other applications include temporary supports to hold an organ, vessel, or tube in a particular position for a controlled, limited time.

These and other modifications and variations to the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention.

That which is claimed is:

1. A method for medical treatment comprising:
applying to tissue, cells, or medical devices an aqueous solution comprising a polymerizable biodegradable polymer mixture, said mixture comprising a first derivatized polysaccharide of dextran and a second derivatized polysaccharide of hyaluronan; and,
polymerizing the derivatized polymers onto a tissue, cell, or medical device wherein the derivatized dextran portion of the resulting gel comprises a water-soluble region and the derivatized hyaluronan comprising a portion that can be degraded by enzymes.

2. The method according to claim 1 wherein the treatment of a medical condition is selectd from the group consisting of a controlled drug delivery, coating an implant, coating cells, coating medical devices for insertion into a patient, and providing a support for tissue.

3. The method according to claim 1 wherein said medical device further comprises vascular grafts, arterial vessels, dilatory stents, and catheters.

4. A hydrogel comprising:
a matrix comprising a plurality of derivatized dextran molecules cross-linked with a plurality of derivatized hyaluronan molecules, wherein a weight ratio of said derivatized dextran molecules to said derivatized hyaluronan molecules ranges from about 20:80 to about 80:20.

5. A hydrogel according to claim 4 wherein said derivatized dextran molecules further comprises an acryloyl-dextran.

6. The hydrogel according to claim 4 wherein said derivatized hyaluronan molecules further comprises an acryloyl-hyaluronan molecule.

7. The hydrogel according to claim 4 wherein said derivatized dextran molecules comprises an acryloyl-dextran molecule and said derivatized hyaluronan molecules comprises an acryloyl-hyaluronan molecule.

8. The hydrogel according to claim 4 wherein said hydrogel further comprises a biological agent physically bound within said matrix.

9. The hydrogel according to claim 8 wherein said biological agent comprises living cells.

10. The hydrogel according to claim 8 wherein said biological agent comprises a pharmacological agent.

11. The hydrogel according to claim 4 wherein said hydrogel further comprises a pharmacological agent covalently bonded to at least one of said derivatized dextran molecules or one of said derivatized hyaluronan molecules.

12. A hydrogel matrix of cross-linked polysaccharides consisting essentially of an acryloyl-dextran cross-linked with an acryloyl-hyaluronan.
